(19) United States
(12) Patent Application Publication
(10) Pub. No.: US 2010/0291197 A1
(43) Pub. Date: Nov. 18, 2010

(54) HOT MELT-FILLED SOFT CAPSULES

(75) Inventor: Ernst Schwab, Wil (CH)

Correspondence Address: THE NATH LAW GROUP, 112 South West Street, Alexandria, VA, 22314, US

(73) Assignee: SWISS CAPS RECHTE UND LIZENZEN AG, Kirchberg (CH)

(21) Appl. No.: 12/733,911

(22) PCT Filed: Sep. 26, 2008
(86) PCT No.: PCT/EP2008/062931

§ 371 Date: Mar. 26, 2010

(30) Foreign Application Priority Data
Mar. 26, 2010 (EP) 07117480

(51) Int. Cl. A61K 31/5513; A61K 31/56; A61K 31/355; A61K 31/192; A61K 31/196; A61K 9/48

(52) U.S. Cl. 424/451; 514/182; 514/458; 514/570; 514/567; 514/221

(57) ABSTRACT

The present invention relates to a soft capsule, comprising a capsule case and a filling material with at least one pharmacologically or physiologically active substance, characterized in that the capsule case is made by melt extrusion of thermoplastically processable polymer material, and the filling material is solid at under 10° C. and has a dripping point of higher than 55° C.

The present invention relates to soft capsules comprising a fill mass solidified at room temperature which is filled into the capsule in the form of a hot molten fill mass, and to a method for the production thereof.

Capsules are established administration forms for medicaments and food supplements. They are designed as core-shell structure, i.e. an ingredient of any desired consistency (the core, also referred to as fill material) is surrounded by a shell made of a suitable coating material. A distinction is made here in particular between hard capsules and soft capsules. Capsules and methods for their production are adequately known (e.g. Stanley, J. P. “Soft gelatin capsules” in: Lachman et al. (Ed.) “The theory and practice of industrial pharmacy”, Philadelphia, Lea & Febiger, 3rd edition (1986); Hofer et al. in: Fahrig, W.; Hofer, U. (Ed.) “Die Kapsel”, Wiss, Verlagsgesellschaft mbH, Stuttgart; Paperback APV volume 7, 1st edition, 1981).

In the case of hard capsules, the coating material consists of a rather thin film (with a thickness up to 200 μm), although this is nevertheless dimensionally stable. As a rule, the shell is composed of two complementary and joinable parts (“body” and “cap”).

The two capsule shell parts are molded in a first step from aqueous solutions of polymers and dried. As a rule, polymers are used which have a sol gel transition state in solution (gelatin, HPMC (hydroxypropylmethyl-cellulose)+carrageenan, etc.). On account of the production technology, only very narrowly restricted compositions of polymer solutions can be used. As a rule, it is not possible to undertake any variations in the composition of the shell, for example in order to subsequently avoid interactions with the fill material, or in order to keep the finished capsules stable in the case of particularly low or particularly high humidity.

In a second step, the upper part is separated from the lower part, the fill material is filled into the lower part and the two shell parts are fitted together again. In the capsule, a gas (air) always remains in the interior of the capsule. In the case of liquid fill materials or fill materials which can become liquid during storage, the gap between the upper and lower parts of the shell has to be sealed using a further film (banding, or spray-film coating) or be stuck using polymeric “glue”.

The shell materials used are usually gelatin, cellulose derivatives, gums, PVA (polyvinyl alcohol), PVP (poly-vinylpyrrolidone) and other synthetic and natural polymers or mixtures of polymers with other substances. Hard capsules are usually filled with powdery, or particulate (pellets) fill materials, more rarely (but also) with liquid, pasty fill materials. In order to avoid the expensive additional sealing or coating, fill materials have in particular also been developed which are incorporated in molten form, are solidified under storage conditions and cannot melt and leak during storage.

Solidification-consolidated melts of lipophilic substances and also highly viscous suspensions of water-soluble particles in lipophilic matrix are suitable for the filling of hard capsules: hard capsules based on gelatin or HPMC comprise hardly any plasticizer and water (aw=0.5, ca. 8% water for gelatin and ca. 4% for HPMC as base material). Consequently, their mechanical properties are also rather tolerant towards lipophilic substances and temperatures up to 80° C. In other words, the shells do not melt or become deformed at temperatures from 25-60° C.

The filling of hard capsules with wax-like substances such as fats, partial glycerides, fatty acid esters (polyethylene glycol fatty acid esters, Gelucire) and long-chain polyethylene glycols (PEG) is known. Such formulations are suitable in particular for the formulation of delayed-release, slow-release pharmaceutical preparations, or for the formulation of sparingly water-soluble active substances in anhydrous, but water-soluble matrices.

In the case of soft capsules, the shell is generally thicker (with a thickness of more than 200 μm). The shell material comprises a plasticizer. Owing to the production method, soft capsules generally consist of one-piece films, i.e. shells which are also tight to nonaqueous liquids. A fill material which has in the past proven suitable is in particular gelatin. The production usually takes place by welding two half-shell films (e.g. Colton process, Upjohn process, Accogel process, Norton process or rotary die process).

In order to ensure the required flexibility of the films during the dripping or the molding and welding, the films consist of aqueous solutions comprising polymer, plasticizer and aggregates. The excess water is removed by drying the molded and filled capsules to the fraction required for storage.

Soft capsules are generally filled with liquid or pasty fill materials (suspension). In principle, they may also be filled—with the help of a special dosing device—with particles (granules or pellets) suspended in liquid phases or with powdery or particulate (pellets) fill materials (without liquid phase). Since hardly any interactions occur between hydrophilic shell and fill material, preference is given to lipophilic liquid fill materials on their own or as carrier matrix for a suspension of crystalline water-soluble substances.

The process parameters (temperature, pressure, time) when incorporating fill materials into soft capsules are determined and/or limited by the conditions which have to be applied for the simultaneous molding and sealing of the two shell parts. For example, in the case of traditional soft gelatin capsule production from aqueous gelatin solutions (by the rotary die process, cf. Bauer in: Fahrig, W.; Hofer, U. (Ed.) “Die Kapsel”, Wiss, Verlagsgessellschaft mbH, Stuttgart; Paperback APV volume 7, 1983, p. 70), on account of the sol gel transition of the aqueous gelatin bands (at ca. 40-45° C.), the temperature that can be tolerated during capsule production is limited to ca. 35° C. (U.S. Pat. No. 6,352,717, col. 1, lines. 60-67; J. P. Stanley, “Soft gelatine capsules”, in Lachmann/Liebermann/Kanig (Ed.), The theory and Practice of Industrial Pharmacy, 1st edition, 1970). Analogously, when using starch/carrageen-water solutions, likewise on account of the sol gel transition (at ca. 50-70° C.), the temperature that can be used when filling the fill material is limited to a maximum of ca. 50° C. Moreover, the water present in the films is often problematic for the fill materials.

For specific compositions, it may be possible to prevent the fill material melts from solidifying through shear viscosity, or to achieve short-term supercooling of the melts prior to incorporation into the soft gelatin capsules by means of special filling devices. Such methods are disclosed in U.S. Pat. No. 6,352,717.

The incorporation of molten fill materials between two encasing bands by means of extrusion/calendering has been described in EP-0 799 012 A1 for producing tablets. However, especially in the case of the calendering of concentrated strands (fill material on the inside, surrounded by outer shell), this technique cannot be referred to as the production of capsule-shaped bodies since the amount of fill material and the film thickness tapers to zero at the edge of the calender mold and therefore in no way can a relatively uniform shell thickness around the molding be achieved. The burrs produced in this way make consumption impossible, and the thinning of the shell makes it difficult to control the pharmaceutical properties (for example in the case of shaping of the shell that is resistant to gastric juices).

The technology of melt extrusion and injection molding is likewise adequately known and has in particular the advantages of being able to use solid solidified melts, thermoplastic polymers and glasses comprising active ingredients in molecularly disperse distribution as fill material. The subsequent covering of such bodies by dipping or spray coating leads to products which, in the technological sense, can likewise not be referred to as capsules.

The production of soft capsules with solid fill materials was thus hitherto limited: filling with particles or powders requires the use of special dosing devices and therefore necessitates modification of the capsule production equipment. By contrast, the filling of soft gelatin capsules with melts is limited, for production-related reasons, to fill materials which have a dripping point at temperatures of up to 35° C. (in the case of starch/carrageen-water solutions for producing the film at up to 50° C.). On the other hand, soft capsules with a solid fill material would be preferred over hard capsules for the reasons discussed above (for example the provision of shells with different mechanical-pharmaceutical properties, and the possibility of minimizing interactions between shell and fill material).

EP-1 437 129 A1 describes a soft capsule made of a gelatin-free material which is filled with a liquid fill mass. This document does not teach filling a fill material with a dripping point above 55° C. into soft capsules of this type.

EP-1 163 901 A1 describes soft capsules made of gelatin. For the reasons stated above and reasons demonstrated by literature (e.g. J. P. Stanley, “Soft gelatine capsules”, in Lachmann/Liebermann/Kanig (Ed.), The Theory and Practice of Industrial Pharmacy, 1st edition, 1970), it is not possible to introduce into capsules of this type a fill material which has a dripping point or melting point of significantly more than 40° C. In this document, exclusively chocolate-like masses are encapsulated which, as is known, melt approximately at body temperature so that they can melt in the mouth. The encapsulation of fill materials with a melting point of about 37° C. in soft gelatin capsules is known and possible. Encapsulation of fill materials with a dripping point above 55° C. is not demonstrated in this document and would also be technically impossible.

EP-0 369 445 A1, WO 93/23022 and WO 99/42086 describe hard capsules with a fill material which must be melted for the filling operation. However, as set forth in detail above, hard capsules can in no way be compared with soft capsules and are not subject to the production-related restrictions with regard to the temperature at which the fill material can be encapsulated.

It was therefore the object of the present invention to provide soft capsules with meltable fill material irrespective of its melting temperature.

This object is achieved according to the invention by a soft capsule comprising a shell and a fill material with at least one pharmacologically or physiologically active substance, characterized in that the shell is composed of polymeric material that can be processed thermoplastically by melt extrusion, and also has a continuous weld seam, and the fill material is solid below 30° C. and has a dripping point of higher than 55° C.

With the present invention, it is possible to transfer the pharmaceutical advantages known from the production of liquid, pasty and melt-filled hard capsules (in particular delayed release and molecular dispersibility) upon the application of pharmaceutical substances to the production of soft capsules and thus to combine advantages of the “melt extrusion” technology for the production of low-water shells without having to accept the complexity of banding or coating as in the case of the production of hard capsules.

The dripping point is the temperature at which the first drop of a melting substance drips from a container under defined conditions. It is a standard method which is described in the European Pharmacopeia (European Pharmacopeia 01/2008:20217, section 2.2.17, pp. 33-34; cf. also Method USP <741> class III). The dripping point is a characteristic parameter for mixtures of substances and, in terms of its weighting, is equivalent to the melting point of pure substances. Pure substances have a sharp, characteristic melting point, whereas mixtures of substances have a more or less broad melting interval which is only partially suitable for characterizing the mixture. The melting behavior of mixtures of substances is therefore better described with the help of the dripping point, which is usually within the melting interval and is a sharp, characteristic parameter of the mixture.

With the present invention, a number of technological obstacles have been overcome which were not able to be solved in the prior art hitherto:
Provision of a heat-tolerant, plastically processible, sealable, water-soluble shell material.
The compatibility of the shell and the active ingredient (fill material) at different temperatures, pressure and water concentrations is achieved.
The different heat expansion coefficients of fill material and shell upon cooling the capsules can be compensated for through additions of a crystalline type.
Avoidance of degassing and the formation of gas bubbles in the fill material (by heating the fill material in the course of a prior heating and vacuum treatment) and between fill material and shell.
Protection of the active ingredients against harmful heating during charging/production.
Variable adjustment of the release behavior (resistance of the shell to gastric juices, immediate or delayed release of the active ingredient).
of the recrystallization of API (Active Pharmacological Ingredient) or fill material matrix made of glassy or partially crystalline systems at temperatures of more than 1-25° C. above the Tg (glass transition point), which is of importance particularly if the Tg is close to zero.

Compared with the prior art, the following advantages arise according to the present invention:
The soft capsule according to the invention is mechanically flexible and more stable than hard capsules. The capsules can not leak.
The soft capsule fill materials according to the invention can be easily formulated so that they are released in a delayed manner.
The mechanical/chemical properties of the shell of the soft capsules according to the invention can be adapted easily to different properties of the fill material or of the storage climate.
The capsule shell can be formed from various thermoplastic materials, in particular also from polymers that are resistant to gastric juices. An additional coating is not necessary for the soft capsules according to the invention.
During the shaping and storage of the capsules, the capsule shell comprises hardly any water (greatly reduced vapor pressure). The fill material compositions therefore hardly change as a result of the passing over of water or plasticizer.
In contrast to hard capsules, the soft capsules according to the invention are completely closed. They are therefore also suitable for the dosing of highly active or toxic substances.
The dripping point of the fill material can—within the range in which fill materials contemplated here by the person skilled in the art having pharmacologically active ingredients melt—be selected upwardly in a practically unlimited manner.
It is possible to use not only fill material matrices based on polymers, but also based on rapidly water-soluble, small molecules which melt and solidify in a glassy crystalline or amorphous manner.
It is possible to eliminate the interaction of shell and fill material since with increasing solidity and increasing uniformity of the phases of the fill material, in particular also the exchange of plasticizer or other small molecules (diffusion) decreases.
The molecular dispersity of fill materials can be obtained with increasing solidity and increasing uniformity of the phases. The recrystallization of the active ingredient is prevented.

The soft capsules according to the invention are characterized by a combination of a specific shell material and a specific fill material.

The shell material is composed of polymeric material that can be processed thermoplastically by melt extrusion. Within the context of the present invention, melt extrusion is to be understood as meaning a preparation, molding and welding of a thermoplastic mass at pressures of more than 1 bar (preferably more than 10 bar, more preferably more than 100 bar) and temperatures of more than 60° C., more preferably more than 80° C., even more preferably more than 100° C.

During the melt extrusion, a band of the corresponding material is formed by converting the material into film form through a flat-film die or with the help of blowing methods at viscosities which do not permit processing while utilizing gravity (casting). Consequently, during the melt extrusion, it is necessary to increase the temperature (by at least 20-50° C.) and also the pressure (to more than 10 or even more than 100 bar) compared to ambient conditions. If appropriate, plasticizers and/or water and also customary additives such as, for example, lubricants, can be added to the thermoplastically processible material.

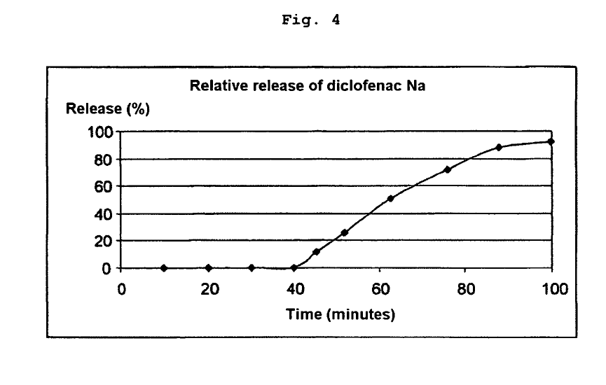

One example of a melt extrusion process that can be used according to the invention is described in EP-1 103 254 A1. The melt extrusion can be carried out in a twin-screw extruder, as described in detail in EP-1 103 254 A1 (working example, FIG. 4). Reference is made expressly to the corresponding disclosure in EP-1 103 254 A1.

According to the invention, the polymeric material that can be processed thermoplastically by melt extrusion can be selected from the group consisting of polyvinyl-pyrrolidone, hydroxypropylcellulose, hydroxypropyl-methylcellulose, polyvinyl alcohol, polyvinyl alcohol acetals, polyethylene glycol-polyvinyl alcohol graft polymer, carbopol, polymer of butyl methacrylate, 2-dimethylaminoethyl methacrylate and methyl methacrylate in the ratio 1:2:1, polyethyl acrylate, methyl methacrylate, polymethacrylic acid, ethyl acrylate, polyethyl acrylate, methyl methacrylate, trimethylammonium ethyl methacrylate chloride, hydroxypropylmethylcellulose acetate succinate, polyox, starch, polylactic acid, polylactic acid coglycolide, gelatin, carrageenan, casein, gluten and mixtures thereof.

Examples of materials suitable according to the invention are the starch-containing masses disclosed in EP-1 103 254 A1, and also the shell materials disclosed in EP-1 586 436 A1, where, in the latter case, shell materials based on PVACL are preferred.

The starch-containing masses described for example in EP-1 103 254 A1 are homogenized, starch-containing masses, comprising preferably at least 45% by weight of an amorphous starch with an amylopectin content greater than or equal to 50% by weight, based on the weight of the anhydrous starch, water, at least one organic plasticizer in a fraction of at least 12% by weight, based on the weight of the anhydrous starch, where the Staudinger index of the homogenized mass is at least 40 ml/g, preferably at least 50 ml/g and even more preferably at least 60 ml/g. According to the invention, the Staudinger index should be understood in the same way as defined in EP-1 103 254 A1. Reference is expressly made to the corresponding disclosure in EP-1 103 254 A1.

The term starch should be understood as meaning native starches, and also physically and/or chemically modified starches. Examples of plasticizers have a solubility parameter of equal to or >16.3 (MPa)^{1/2}. The plasticizers can be selected from the group consisting of polyalcohols, organic acids, amines, acid amides and sulfoxides. Preference is given to polyalcohols. Glycerol has proven to be the most suitable plasticizer. In one preferred embodiment, the content of organic plasticizers is in the range from 30% by weight to 50% by weight, preferably in a range from 38% by weight to 45% by weight.

In a further embodiment, the mass additionally comprises a lubricant and mold release agent which is selected from the group consisting of lecithins, mono-, di- and triglycerides of food fatty acids, sugar esters of food fatty acids and food fatty acids.

In a further embodiment, the mass can additionally also comprise at least one aggregate in a weight range from 3.5% by weight to 15% by weight, based on the total weight of the mass, preferably from 5% by weight to 8% by weight. The aggregate is determined by the required properties of the molding produced from the homogenized mass and is selected from the group consisting of carbonates and/or hydrogencarbonates of the alkali metal ions and/or alkaline earth metal ions, further disintegration aids, dyes, preservatives, antioxidants, physically and/or chemically modified biopolymers, in particular polysaccharides and vegetable polypeptides. As disintegration agents for one-part capsule shells, preference is given to using calcium carbonate and amylases.

Alternative materials that can be used are described in EP-0 090 600 A1. Reference is expressly made to the corresponding disclosure in EP-0 090 600 A1.

According to the invention, it is also possible to use the thermoplastic polymers described in EP-1 586 436 A1, which are selected from the group consisting of polyvinyl alcohol, cellulose ethers, polycaprolactone, polyamides, polyacrylic acid, poly-vinylpyrrolidone, polylactic acid or polyvinyl alcohol acetals (PVACL), derivatives or mixtures thereof. Reference is expressly made to the corresponding disclosure of EP-1 586 436 A1.

According to the invention, the shell preferably consists of a thermoplastic polymer water-soluble at 37° C.

According to the invention, the shell further preferably consists of polymers which are resistant to gastric juices or dissolve depending on the pH.

From these materials it is possible to produce, by melt extrusion, films which can be processed in customary soft capsule production methods, such as, for example, the rotary die method, to give soft capsules. Preferably, the two steps (melt extrusion and encapsulation) are carried out in-line (i.e. continuously). The present invention relates to methods in which two films are sealed together with formation of a continuous weld seam. The method most customary for this is the rotary die method.

The rotary die method is generally known and described, for example, in EP-1 103 254 A1. Reference is expressly made to the corresponding disclosure in EP-1 103 254 A1.

According to the present invention, the incorporation of the fill material in the capsule molding step (e.g. the rotary die method) likewise takes place at the elevated temperatures which are used in the preceding step of melt extrusion. The subsequent welding of the films to form the capsule likewise takes place under the elevated temperature and pressure conditions stated for the melt extrusion. According to the invention, a pressure is generally to be applied which exceeds the pressure in a conventional rotary die process by a factor of 10. According to the invention, preference is given here to pressures which exceed the pressure in a conventional rotary die process by a factor of 15 to 40, i.e. are approximately in the pressure range from 225 to 600 bar.

According to the invention, particularly preferably thermoplastic polymers can be used whose viscosity curves, at a temperature below the liquidus melting temperature in the range from 100 to 750 bar, have an increase DELTA eta/DELTA P of at least −30 Pas/bar and of at most −100 Pas/bar. The polymers preferably fulfill this property at a temperature in the range 50-180° C.

According to the invention, the capsule is preferably quenched following production in order to make the fill material glassy. This takes place preferably by cold gases (nitrogen, air, CO₂) or by a cold bath of liquids compatible with the shell.

Surprisingly, the shell materials used according to the invention have proven to be suitable for producing soft capsules which are filled with molten fill material. In particular, the shell materials used according to the invention can be subjected during the capsule production to significantly higher temperatures than the gelatin-based materials primarily used hitherto. Consequently, the soft capsules according to the invention can be filled with fill materials which have a dripping point of higher than 55° C. Here, the fill material can be introduced at similar temperatures to those given above for the melt extrusion. A thermally insulated filling wedge (as is disclosed, for example, in EP-1 216 680 A1) in the rotary die apparatus is advantageous here, the fill material in this case preferably being dosed at a lower temperature than the encapsulation temperature.

Upon contact with water, the fill materials according to the invention exhibit adequate potential bioavailability. “Potentially adequate bioavailability” is understood to mean a molecularly disperse supply of the API in the gastrointestinal tract that is immediate or delayed and adapted to the pharmacological effect and the physiological conditions. For good solubility in water of the substance, at least 80% of the API should be dissolved within the required time in the liquids of the gastrointestinal tract, which can be measured as simulation with the so-called release. For sparingly water-soluble or lipophilic, water-immiscible API, this is understood as meaning the emulsification of at least 80% of the API in the form of droplets, colloidal or other complicated structures with a surface area at least 1000 times the original “capsule fill material surface area”.

The transition from the “solid” state to the “plastic” or “liquid” state can in the case of low molecular weight and crystallizing substances be described by the so-called melting point (m.p.). The melting temperature is the term used to refer to the temperature at which a substance melts, i.e. converts from the solid state to the liquid state. The melting temperature is dependent on the substance, but, in contrast to the boiling temperature, is only very slightly dependent on the pressure (melting pressure). Melting temperature and melting pressure are referred to together as melting point, where this describes the state of a pure substance and is part of the melting curve in the phase diagram of the substance. For pure substances, the melting point is identical to the freezing point and remains constant throughout the entire melting process.

For polymers, amorphous or glassy substances, the transition temperature or glass temperature is characteristic. The glass transition temperature or softening temperature (Tg) is the temperature at which a glass has the largest change in deformability. This so-called glass transition separates the lower brittle energy-elastic range (=glass range) from the upper soft entropy-elastic range (=rubber-elastic range). The transition in the flow range of the amorphous plastic is fluid. Partially crystalline plastics have both a glass transition temperature, below which the amorphous phase “freezes” (associated with embrittlement), and also a melting temperature, at which the crystalline phase melts. The melting temperature clearly separates the entropy-elastic range from the flow range. In contrast to this, crystalline plastics have only one melting temperature.

For blends and mixtures of all types, the so-called dripping point is best suited for describing the behavior. This is the temperature at which the fill material drips under the influence of gravity (e.g. method USP <741> class III), as has already been explained above. This dripping point is independent of when the “first” or “last” component is “liquid”, but is governed only by the “average” behavior.

Rotary die soft capsule technology meters the fill material by means of piston pumps. It is therefore necessary for the fill material to be pumpable at the processing temperature. The viscoelastic behavior of a fill material must correspond to a viscosity of less than 50 Pas, more preferably less than 10 Pas, even more preferably less than 5 Pas. It is unimportant whether the fill material mixture takes place batchwise by melting, mixing, cooling and reheating, or continuously directly by melting, mixing and incorporation into the capsule. Similarly, conventionally the production in the heated mixer or by means of an extruder can be used analogously for the process.

According to the invention, the fill material is preferably prepared continuously directly prior to the encapsulation from the corresponding starting components by melting and mixing. Preferably, at least one active ingredient is continuously metered in solid form into the fill material melt and dissolved or ground in. Particularly preferably, the active substance is added in microparticulate form to the melt. According to the invention, the fill material is particularly preferably prepared continuously with the help of an extruder.

Surprisingly, it has been found that for most customary pharmaceutical, “nutritional” active ingredients (vitamins, mineral substances etc.) (here generally called API), the temperature-time profile, which arises from using rotary die encapsulation at relatively high temperatures, remains without negative effects. This is the case in particular if
a) the individual raw material components have been melted under reduced pressure and/or protective gas
b) mixing of the components takes place at the last moment prior to incorporation into the capsule (of particular suitability is a continuous melting, mixing and degassing process as in a single-screw or twin-screw extruder)
c) the incorporation into the capsules takes place at the lowest possible temperature through a filling-material line through a filling wedge, which is insulated from the heating of the thermoplastic shell material
d) rapid cooling of the prepared capsules takes place
e) the thermoplastic shell material is tolerant to softening and quenching cycles
f) water vapor pressure and/or water concentration during the encapsulation and cooling operations is taken into account (low-water procedure).

Surprisingly, it has likewise been found that in the case of a suitable embodiment of fill materials that are solid at about 21° C. to 30° C., which are pumpable at temperatures of preferably above 48° C. and can be filled into soft capsules with suitable shell materials and a suitable molding-encapsulation process, an entirely normal release can be achieved. Normal release (neither rapid (within minutes) nor delayed (within hours)) is typically to be understood as meaning a release of more than 80% of the API within 30 to 60 minutes. Such a preparation comprises the API in a solid lipophilic self-emulsifying preparation (SSEP solid self-emulsifying phase).

The admixing of a further solid (polymeric, crystalline or amorphous) hydrophilic phase (HSP), which is essentially insoluble in the self-emulsifying phase (SSEP) itself at the filling temperature of the preparation has proven particularly suitable. Said phase can be selected from crystalline or glassy water-soluble substances or water-insoluble polymeric (fibrous) substances which, in a type of wick effect, draw water into the preparation or, as a result of rapid dissolution, allow erosion and distribution of SSEP fragments.

In a particularly advantageous preparation, the carrier for the API consists directly of such water-soluble amorphous or partially crystalline melts of these substances.

Suitable matrices are substances which, on their own or in a mixture, have a dripping point of more than 40° C. and are acceptable pharmaceutically or as a food additive for oral consumption.

By way of example, mention may be made here of:
“Soft melt matrix”: wax-like substances which melt or soften between 30 and 90° C. and in particular also substances which dissolve or emulsify in water: poloxamers (polyoxyethylene-polyoxy-propylene block polymers), sorbitan esters (Span), polyethylene-sorbitan-fatty acid esters (e.g. Tween=polysorbate 65), vegetable and animal fats (e.g. mono- or di- or triglycerides, e.g. hydrogenated cottonseed oil), vegetable and synthetic waxes (e.g. beeswax, microcrystalline waxes), tocopherol esters (e.g. vitamin succinate, vitamin E TPGS), polyols (polyethylene glycol >1500), sucrose fatty acid esters (e.g. sucrose palmitates), propylene glycol fatty acid esters (e.g. propylene glycol palmitostearate), ethylene glycol fatty acid esters (e.g. ethylene glycol palmitostearate), polyglycerol fatty acid esters (e.g. decaglycerol distearate), fatty acid esters of acetic acid, citric acid, lactic acid and tartaric acid (e.g. Imwitor 2020).
Water-soluble crystalline or glassy substances, essentially anhydrous or comprising water up to the use equilibrium moisture (aw<0.65): isomaltol, sorbitol, maltitol, erythritol, mannitol, xylitol, hydrogenated hydrolysis products of starch (polyols and polyol ethers), maltodextrin, dextrin, soluble starch, sucrose, glucose, fructose etc., and mixtures thereof with one another.
Water-soluble amphoteric polymers for thickening: PVP (polyvinylpyrrolidone), acrylic or methacrylic polymers (Eudragit), PEG-PVA graft polymers (Kollicoat).
Solvents/plasticizers: glycerol, propylene glycol, ethyl diglycol, salts (NaCl), sodium sulfate, calcium sulfate.
Water-insoluble and polymeric aggregates: microcrystalline cellulose, crosslinked polyvinyl-pyrrolidone, crosslinked sodium carboxymethyl-cellulose, insoluble (native) starches, cyclodextrins etc.

According to the invention, the fill material can comprise the following active ingredients listed by way of example and not exhaustively:
self-meltable API: fatty acids, vitamin succinate, vitamin E nicotinate, carotenoids-fatty acid esters (e.g. lutein ester), phytosterols (m.p. 80-140° C.), phytosterol esters (m.p.)50°, lecithins (phosphatidylcholine), propranolol (m.p. 96° C.), chlorate hydrates (m.p. 57° C.), chlor-amphenicol palmitate (m.p. 90° C.), cloforex (m.p. 53° C.), cyclandelate (m.p. 53° C.) etc.

API embedded in melt matrix:
API which are soluble or dispersible in SSEP with heating, but only crystallize to a limited extent, if at all, in the matrix: for example ibuprofen, diclophenac sodium, acyclovir
API of complex composition, where certain fractions are dissolved, and others are only suspended. To be mentioned here in particular are plant extracts of highly diverse origin and concentration, which may be carrier-containing or carrierless, spissum or dry.

The soft capsules according to the invention can comprise customary additives. For example, inert crystalline aggregates such as calcium triphosphate are suitable for reducing the difference in the heat expansion of the molten filling matrix compared with the thermoplastic mixture of the shell during the solidification operation, such that partial separation of the capsule shell from the contents does not result.

According to the invention, extracts are to be understood as meaning all extracts from plant material of any plant organs with partial omission of primary plant ingredients such as cellulose, saccharides, proteins or triglycerides. The purpose of these extracts consists in enriching certain secondary plant ingredients, in particular those with desired pharmacological properties. Comminution of the plant material, disruption of the plant cells by grinding, heating, ultrasound treatment, and also the extraction, enrichment through evaporation of the solvent and purification are adequately known to the person skilled in the art.

According to the invention, use equilibrium moisture is the product moisture which is established when the capsule, by being left to stand, is brought to an equilibrium with the ambient moisture (air) (average kinetic moisture climatic zone I: ca. 45% RH, product equilibrium moisture aw=0.45, absolute water content dependent on the sorption of the product).

The release properties of the soft capsules are determined in the present invention in accordance with USP method <724>.

According to the present invention, “molecularly disperse” is understood as meaning a solution or solution-like to colloidal distribution of the API which is essentially free from macroscopically ascertainable concentration accumulations of the API in crystalline or amorphous form. The molecularly disperse phase may be liquid (solution), semisolid (solid and liquid present together) or solid (glass or partially crystalline/crystalline).

The soft capsules according to the invention can be used for all conventional purposes for which soft capsules are used. By way of example, mention may be made of oral, rectal or vaginal application, where the capsule can be applied as food supplement, pharmaceutical product, medicinal product or for cosmetic or technical purposes.

The present invention is illustrated below by reference to nonlimiting examples and figures. These show:

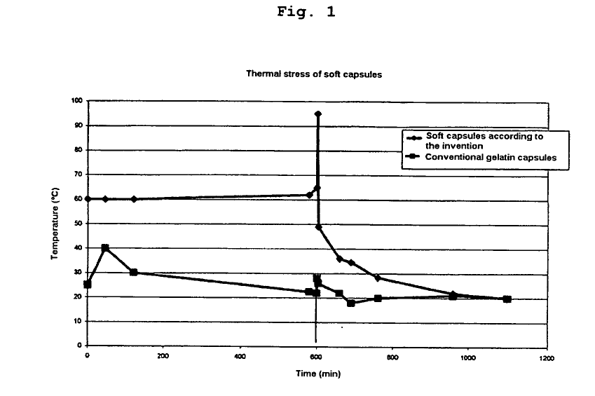

FIG. 1: the temperature profile of a rotary die encapsulation for conventional gelatin capsules and for the soft capsules according to the invention

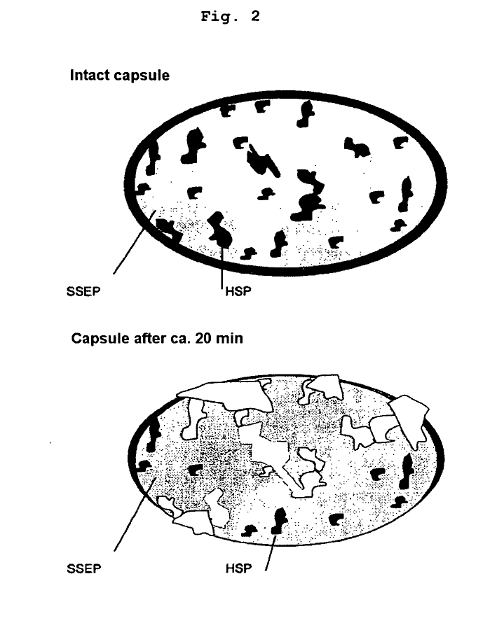

FIG. 2: an embodiment according to the invention of a soft capsule with the wick effect achieved by adding HSP

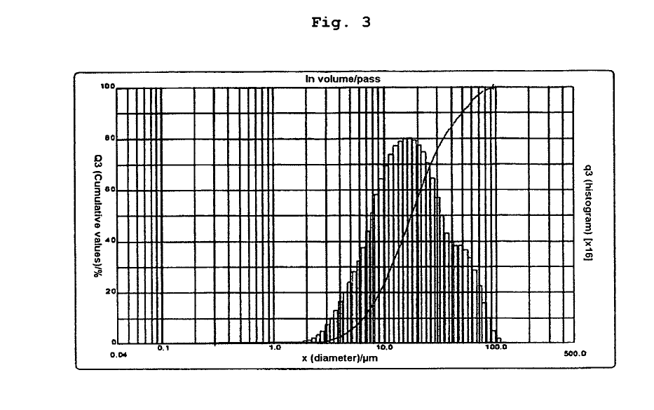

FIG. 3: the droplet distribution following release of the fill material from a soft capsule as in example 2

FIG. 4: the release rate of diclofenac from a soft capsule as in example 4

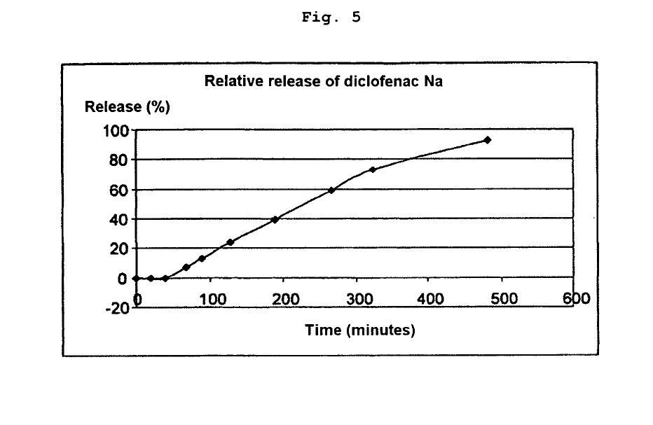

FIG. 5: the release rate of diclofenac from a soft capsule as in example 5

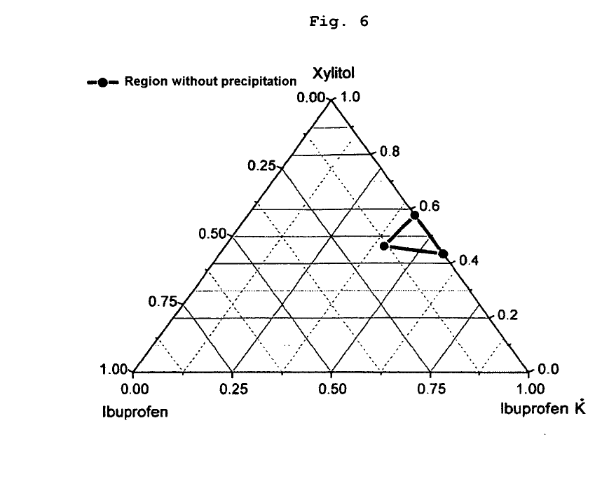

FIG. 6 a diagram with the glass-like states suitable according to the invention between ibuprofen K (prepared from titration of ibuprofenic acid and KOH), ibuprofen and the matrix xylitol.

It is evident from FIG. 1 that in the case of the present invention a considerably higher temperature profile can be applied than in the case of conventional soft capsules made of gelatin. It is thus possible to also provide filling materials with a higher dripping point (i.e. above 55° C.) in soft capsules.

FIG. 2 shows a preferred embodiment of the soft capsule according to the invention. Here, the filling material comprises an above-described self-emulsifying phase (SSEP) and also a likewise above-described, admixed further solid hydrophilic phase (HSP). Upon adding the capsule to an aqueous medium, the HSP draws water into the preparation in a type of wick effect. Consequently, the rate of disintegration of the capsule according to the invention is significantly increased.

### EXAMPLE 1

180 mg of a purified fractionated lipophilic extract from plants, in particular soya beans, comprising more than 90% of sterols with a melting range of 135-145° C., were melted together with 90 mg of polysorbate 65 (m.p. ca. 31° C.) and 5 mg of sucrose monodipalmitate (m.p. 49-55° C.) with vigorous stirring at 150° C. for 10 min and then rapidly cooled. The wax-like solid mixture with a dripping point of >55° C. was again heated to 65° C. and filled, using dosing pumps, in amounts of in each case 275 mg, into soft capsules with a shell made of starch, sorbitol, maltitol, glycerol and glycerol monostearate (VegaGel®). The technology of melt extrusion production of the capsule shell band was used. The sealing temperature of the shell seam was 95° C. The capsules reached temperatures around 75° C., and were then cooled with air at 20° C. The capsules disintegrated in water at 37° C. within several hours to form an emulsion/suspension.

### EXAMPLE 2

250 mg of vitamin E succinate (D-a-tocopherol succinate, m.p. 76° C.) were melted and intensively mixed with 70 mg of Cremophor RH40 (Cognis, polyoxy-40-hydrogenated castor oil USP) at 80° C. under nitrogen. In each case, 320 mg of the resulting slightly cloudy mixture with a dripping point of above 55° C. were filled at 60° C. into soft capsules with a shell made of polyvinyl alcohol-starch-acetal (PVACL, Natutec™) with glycerol plasticizer. The technology of melt extrusion production of the capsule shell band was used. The sealing temperature of the shell seam was 130° C. The capsules reached temperatures of ca. 85° C., were then cooled with air at 20° C. and, after 5 minutes, had a temperature of less than 40° C. The capsules were storage-stable and disintegrated in water at 37° C. within 45 min. The capsules were in fact so resistant to gastric juices that they would only open in the small intestine. The fill material initially floated. Over a period of 2.5 h, the vitamin E converted to an emulsion with an average particle size (“droplet size”) of 15-20 μm. The resulting droplet distribution is shown in FIG. 3.

### EXAMPLE 3

A cold mixture of 200 mg of xylitol (m.p. 93° C.), 200 mg of ibuprofen potassium salt, 24 mg of water and 24 mg of PVP (Kollidon K12) were homogeneously melted in a 1-shaft extruder and passed directly to a capsule filling at 90° C. In the rotary die process, a polyvinyl alcohol starch acetal (PVACL, Natutec™) was used as shell material for the 7.5 oval capsule with fill weight 448 mg. The sealing temperature was ca. 130° C. The capsule was introduced directly after production into cold paraffin (decane) at ca. 0° C. The capsule contents still exhibited no crystallization phenomena after 6 weeks. Starting at 40 min with opening of the capsule, more than >90% of the active ingredient were released within 30 minutes. FIG. 6 shows a diagram which illustrates the usable glass-like states between ibuprofen K (prepared from titration of ibuprofenic acid and KOH), ibuprofen and the matrix xylitol.

### EXAMPLE 4

At ca. 65° C., 75 mg of diclofenac sodium (m.p. 283° C.) in finely crystalline form were introduced into a melt of 75 mg of Lutrol F127 (poloxamer, polyoxyethylene-polyoxypropylene block polymer, BASF) (m.p. 53-57° C.), 451 mg of Gelucire 44/14 (PEG-32 glyceryl laurate, Gattefosse (m.p. 44° C.)), and the mass was filled at 60° C. into cans each of 601 mg in 11 minims oblong soft capsules with a shell made of polyvinyl alcohol-starch acetal (PVACL, Natutec™). The technology of melt extrusion production of the capsule shell band was used. The sealing temperature of the shell seam was 130° C. The capsules reached temperatures of ca. 85° C., were cooled with air at 20° C. and, after 5 minutes, had a temperature of less than 40° C. The capsules were storage-stable and disintegrated in water at 37° C. within 45 min. The capsules were in actual fact so resistant to gastric juices that they would only open in the small intestine. After a delay of 45 min, diclofenac was released to around 95% within 1 hour. The corresponding results are shown in FIG. 4.

### EXAMPLE 5

At ca. 65° C., 75 mg of diclofenac sodium (m.p. 283° C.) in finely crystalline form were introduced into a melt of 385 mg of Lutrol F127 (poloxamer, polyoxyethylene-polyoxypropylene block polymer, BASF) (m.p. 53-57° C.), 385 mg of Gelucire 50/13 (stearoyl macrogol glycerides, Gattfossé (m.p. 50° C.), and the mass was filled at 60° C. into 14 minims oblong soft capsules with a shell made of polyvinyl alcohol-starch acetal (PVACL, Natutec™) The technology of melt extrusion production of the capsule shell band was used. The sealing temperature of the shell seam was 130° C. The capsules achieved temperatures of ca. 85° C., were cooled with air at 20° C. and, after 5 minutes, had a temperature of less than 40° C. The capsules were storage-stable and disintegrated in water at 37° C. within 45 min. The capsules were in fact so resistant to gastric juices that they would only open in the small intestine. After a delay of 45 min, diclofenac was released to around 95% within 7 hours. The corresponding results are shown in FIG. 5.

### EXAMPLE 6

10 mg of temazepam were dissolved in a melt of 250 mg of PEG20000 (ca. 60° C.) at 65° C. To establish the correct equilibrium moisture (aw=0.45), 1.4 mg of water (0.55%) were added to the mass, and, in amounts of in each case 262 mg, the mass was filled into soft capsules with a shell made of starch, sorbitol, maltitol, glycerol and glycerol monostearate (VegaGel™). The technology of melt extrusion production of the capsule shell band was used. The sealing temperature of the shell seam was 95° C. The capsules achieved temperatures of ca. 73° C., were then cooled with air at 20° C. and, after 3 minutes, had a temperature of less than 40° C. The capsules were storage-stable and disintegrated in water at 37° C. within 20 min. The molecularly disperse fill material dissolved completely within 15 minutes.

1.-19. (canceled)

20. A soft capsule comprising a shell and a fill material with at least one pharmacologically or physiologically active substance, wherein the shell is composed of polymeric material that can be processed thermoplastically by melt extrusion, and also has a continuous weld seam, and the fill material is solid below 30° C. and has a dripping point of higher than 55° C.

21. The soft capsule as claimed in claim 20, wherein the polymeric material that can be processed thermoplastically by melt extrusion is selected from the group consisting of polyvinylpyrrolidone, hydroxypropylcellulose, hydroxypropylmethylcellulose, polyvinyl alcohol, polyvinyl alcohol acetals, polyethylene glycol-polyvinyl alcohol graft polymer, carbopol, polymer of butyl methacrylate, 2-dimethylaminoethyl methacrylate and methyl methacrylate in the ratio 1:2:1, polyethyl acrylate, methyl methacrylate, polymethacrylic acid, ethyl acrylate, polyethyl acrylate, methyl methacrylate, trimethylammonium ethyl methacrylate chloride, hydroxypropylmethylcellulose acetate succinate, polyox, starch, polylactic acid, polylactic acid coglycolide, gelatin, carrageenan, casein, gluten and mixtures thereof.

22. The soft capsule as claimed claim 20, wherein the polymeric material that can be processed thermoplastically by melt extrusion is a homogenized, starch-containing mass.

23. The soft capsules as claimed in claim 22, wherein the polymeric material that can be processed thermoplastically by melt extrusion is a homogenized, starch-containing mass comprising at least 45% by weight of an amorphous starch with an amylopectin content greater than or equal to 50% by weight, based on the weight of the anhydrous starch, water, at least one organic plasticizer in a fraction of at least 12% by weight, based on the weight of the anhydrous starch, wherein the staudinger-index of the homogenized mass is at least 40 ml/g.

24. The soft capsules as claimed in claim 23, wherein the Staudinger index of the homogenized mass is at least 50 ml/g

25. The soft capsules as claimed in claim 23, wherein the Staudinger index of the homogenized mass is at least 60 ml/g.

26. The soft capsule as claimed in claim 20, wherein the polymeric material that can be processed thermoplastically by melt extrusion comprises additional substances selected from the group consisting of plasticizers, dyes, UV stabilizers, preservatives, antioxidants, physically and/or chemically modified biopolymers, disintegration accelerators, shape stabilizers, lubricants and mold release agents and separating agents.

27. The soft capsule as claimed in claim 20, wherein the pharmacologically or physiologically active substance is an active ingredient that is in molecularly disperse distribution.

28. The soft capsule as claimed in claim 20, wherein the fill material is glass-like, partially crystalline or crystalline.

29. The soft capsule as claimed in claim 20, wherein the fill material comprises at least one active-ingredient-containing melt and additionally active-ingredient-carrying particles.

30. The soft capsule as claimed in claim 20, wherein the fill material comprises a pharmacologically or physiologically active substance in a solid lipophilic self-emulsifying preparation.

31. The soft capsule as claimed in claim 30, wherein the fill material comprises a further solid hydrophilic phase which is essentially insoluble in the self-emulsifying phase itself at the filling temperature of the preparation.

32. A method of producing soft capsules as claimed in claim 20, comprising the steps
a) preparation of a shell material from a thermoplastically processible polymeric material by melt extrusion
b) provision of a fill material by melting such that it is pumpable,
c) producing soft capsules by the rotary die method, where the molten fill material is metered into the soft capsule and the capsule is then cooled to ambient temperature.

33. The method as claimed in claim 32, wherein the capsule is quenched following production in order to make the fill material glassy.

34. The method as claimed in claim 33, wherein the quenching takes place by cold gases or by a cold bath of liquids compatible with the shell.

35. The method as claimed in claim 32, wherein the fill material is metered through a filling wedge insulating the fill material at a lower temperature than the encapsulation temperature.

36. The method as claimed in claim 32, wherein the fill material is prepared continuously directly prior to encapsulation from the corresponding starting components by melting and mixing.

37. The method as claimed in claim 32, wherein at least one active ingredient in solid form is continuously metered and dissolved or ground into the fill-material melt.

38. The method as claimed in claim 32, wherein the active substance is added in microparticulate form to the melt.

39. The method as claimed in claim 32, wherein an extruder is used for the continuous preparation of the fill material.
